(19) United States
(12) Patent Application Publication
(10) Pub. No.: US 2025/0072823 A1
(43) Pub. Date: Mar. 6, 2025

(54) A METHOD AND DEVICE FOR TESTING MUSCLE STIFFNESS OR SPASTICITY

(71) Applicants: Nanyang Technological University, Singapore (SG); Tan Tock Seng Hospital Pte Ltd, Singapore (SG)

(72) Inventors: Dace GAO, Singapore (SG); Jian CHEN, Singapore (SG); Kaushik PARIDA, Singapore (SG); Pooi See LEE, Singapore (SG); Keng He KONG, Singapore (SG)

(21) Appl. No.: 18/729,616

(22) PCT Filed: Jan. 20, 2023
(86) PCT No.: PCT/SG2023/050043

§ 371 Date: Jul. 17, 2024

(30) Foreign Application Priority Data
Jan. 21, 2022 (SG) 10202200642W

(51) Int. Cl. A61B 5/00; A61B 5/11

(52) U.S. Cl. CPC A61B 5/4519; A61B 5/1107

(57) ABSTRACT

A device and method of testing muscle stiffness or spasticity. The method includes: controlling an actuator to periodically change between a pressurized state and a relaxed state such that the actuator in at least the pressurized state provides a cyclic force contributing to at least one deformation of a piezoelectric film, the actuator in at least the relaxed state providing a lateral surface in abutment with the piezoelectric film, the piezoelectric film being variably deformable and conformable to the muscle or the muscle group; acquiring a signal generated by the at least one deformation of the piezoelectric film, wherein a change in the signal corresponds to the at least one deformation of the piezoelectric film; and determining a quantitative measure of a muscle spasticity state of the muscle or the muscle group based on the signal.

The present application claims priority to the Singapore patent application no. 10202200642W filed on Jan. 21, 2002, the contents of which are incorporated in entirety by reference.

### TECHNICAL FIELD

The present disclosure relates to the field of diagnosis or testing of muscle-related physiological or biomedical conditions, and more particularly to a method of testing the stiffness of a muscle or a muscle group.

### BACKGROUND

Spasticity is a neurological symptom featuring an abnormal increase in muscle tone. It may be associated with neurological disorders in central nervous system that undermines the voluntary control in muscles. The most common disorders that cause spasticity include stroke, cerebral palsy, spinal cord injury, traumatic brain injury, multiple sclerosis, and haemorrhage, etc. Spasticity not only impairs a patient's mobility in daily life, but also introduces other illnesses such as muscle pain, joint contractures, and pressure ulcers. In order to relieve spasticity and restore normal control in muscles, pharmacological and surgical therapies have been developed. However, a major limitation in spasticity treatments is the lack of instruments or methods which can quickly, consistently, and more precisely evaluate the severity of spasticity.

### SUMMARY

According to one aspect, the present application discloses a method of testing a muscle or a muscle group, the method comprising: controlling an actuator to periodically change between a pressurized state and a relaxed state such that the actuator in at least the pressurized state provides a cyclic force contributing to at least one deformation of a piezoelectric film, the actuator in at least the relaxed state providing a lateral surface in abutment with the piezoelectric film, the piezoelectric film being variably deformable and conformable to the muscle or the muscle group; acquiring a signal generated by the at least one deformation of the piezoelectric film, wherein a change in the signal corresponds to the at least one deformation of the piezoelectric film; and determining a quantitative measure of a muscle spasticity state of the muscle or the muscle group based on the signal.

According to the method, the piezoelectric film may be conformable to both the actuator in the pressurized state and to the muscle or the muscle group. Preferably, the piezoelectric film is flexible, and the piezoelectric film is variably deformable in response to a stiffness of the muscle or the muscle group. The piezoelectric film may be held in a test position independently of the cyclic force, the test position being between the lateral surface and the muscle or the muscle group.

The method may further comprise controlling a fluid communication between a fluid source and an elastic bag of the actuator, wherein the lateral surface is part of the elastic bag, and wherein the actuator is in the pressurized state if the elastic bag is inflated by the fluid source, wherein the actuator is in the relaxed state if the elastic bag is at least partially deflated.

According to the method, the signal may be a time series voltage signal comprising at least one local peak corresponding to the pressurized state. According to the method, a mean amplitude of the at least one local peak may correspond to a degree of severity of a muscle spasticity state.

According to the method, the signal may be acquired when the muscle or the muscle group is in an isometric contraction. According to the method, the signal may be acquired when the muscle or the muscle group is in a static state.

According to the method, the signal may include at least one first local peak if the muscle or the muscle group is in a flexion state, and the signal may include at least one second local peak if the muscle or the muscle group is in an extended state.

The method may further comprise: determining the muscle spasticity state based on a ratio between the at least one first local peak and the at least one second local peak. The method may further comprise: determining the muscle spasticity state based on a difference between the at least one first local peak and the at least one second local peak. The method may further comprise: determining a correlation table based on at least one of the following: (i) at least one local peak of the signal, (ii) a mean of plurality of local peak of the signal, (ii) a ratio between at least one first local peak of the signal and at least one second local peak of the signal, (iii) or any combination thereof. The quantitative measure may correlate to a Modified Ashworth Scale (MAS) value.

The method may further comprise: periodically switching the actuator between the pressurized state and the relaxed state; and concurrently acquiring the signal from the piezoelectric film when the muscle is in a transition state, the transition state being characterized by a dynamic motion of the muscle or the muscle group between a flexion state of the muscle and an extended state of the muscle.

According to the method, the actuator may be configured to switch between the pressurized state and the relaxed state at a frequency within a frequency range.

According to the method, the signal may comprise a time series of peaks, the signal including at least one first local peak corresponding to the flexion state, at least one second local peak corresponding to the extended state, and at least one third local peak corresponding to the transition state. The method in which the at least one third local peak may be characterized by a smaller voltage value than each of the at least one first local peak and the at least one second local peak. The method in which a difference between the at least one third local peak and the at least one first local peak corresponds to a muscle spasticity state.

In another aspect, the present application discloses a device for testing a muscle or a muscle group, the device comprising: a piezoelectric film; and an actuator, the actuator in at least a relaxed state having a lateral surface in abutment with the piezoelectric film, the actuator being operable to periodically change between a pressurized state and the relaxed state such that the actuator in at least the pressurized state provides a cyclic force contributing to at least one deformation of the piezoelectric film, the piezoelectric film being variably deformable and conformable to the muscle or the muscle group, wherein the at least one deformation of the piezoelectric film is configured to generate a signal, and wherein a change in the signal corresponds to the at least one deformation of the piezoelectric film.

The piezoelectric film may be conformable to both the actuator in the pressurized state and to the muscle or the muscle group.

Preferably, the piezoelectric film is flexible, and the piezoelectric film is variably deformable in response to a stiffness of the muscle or the muscle group.

The piezoelectric film may be configured to provide a first signal representative of a first curvature change of the piezoelectric film when the muscle is in flexion and a second signal representative of a second curvature change of the piezoelectric film when the muscle is in extension.

The device may further comprise a wearable article, wherein the piezoelectric film is held by the wearable article in a test position independently of the cyclic force, the test position being between the lateral surface and the muscle or the muscle group. The wearable article may further comprise a pocket, wherein the actuator and the piezoelectric film are disposed in the pocket.

The actuator may comprise an elastic bag, the lateral surface being a part of the elastic bag, in which the lateral surface is larger than a surface of the piezoelectric film, and in which the elastic bag is disposed with the lateral surface adjacent to a surface of the piezoelectric film.

The piezoelectric film may have a higher tension stiffness along a neutral plane of the piezoelectric film, and the piezoelectric film may have a lower bending stiffness about a bending axis in the neutral plane of the piezoelectric film.

In yet another aspect, the present application discloses a system for testing a muscle or a muscle group, the system comprising: a wearable article; a piezoelectric film; an actuator, a controller, and a fluid source coupled to the controller. The actuator is attachable to the wearable article such that the actuator in at least a relaxed state provides a lateral surface in abutment with the piezoelectric film. The piezoelectric film is disposed between the actuator and the muscle or the muscle group such that the actuator in at least the pressurized state provides a cyclic force periodically conforming the piezoelectric film with the muscle or the muscle group. The piezoelectric film is configured to generate a signal corresponding to at least one deformation of the piezoelectric film, the signal being generated concurrently with the actuator periodically changing between the pressurized state and the relaxed state. The controller is configured to open or close a fluid communication between the fluid source and the actuator such that the actuator is periodically changed between the pressurized state and the relaxed state.

Preferably, the actuator comprises an elastic bag, wherein the actuator is in the pressurized state if the elastic bag is inflated by the fluid source, and wherein the actuator is in the relaxed state if the elastic bag is at least partially deflated.

### BRIEF DESCRIPTION OF DRAWINGS

FIG. 1 is a schematic view of a system for testing or measuring a state or a condition of a muscle or a muscle group according to an embodiment of the present disclosure;

FIG. 2 is a perspective view of a device according to an embodiment of the present disclosure;

FIG. 3 is a sectional view A-A of the device according to FIG. 2;

FIG. 4 is a partial exploded view of the device according to FIG. 2;

FIG. 5 is a side view of the device of FIG. 4;

FIG. 6 is a partial sectional view showing an actuator of the present device in a relaxed state;

FIG. 7 is a partial sectional view of the actuator of FIG. 6 in a pressurized state;

FIG. 8 is a partial sectional view of the actuator in a relaxed state according to another embodiment;

FIG. 9 is a partial sectional view of a device according to yet another embodiment with the actuator in a relaxed state;

FIGS. 10A and 10B are schematic views of fluid communication paths between a fluid source and an actuator according to an embodiment of the present disclosure;

FIGS. 11A and 11B are schematic views of fluid communication paths between a fluid source and an actuator according to another embodiment of the present disclosure;

FIG. 12 is a flow chart of a method for diagnosing a condition of a muscle or a muscle group according to an embodiment of the present disclosure;

FIG. 13 is an exemplary signal obtained from measuring a subject's bicep in an extended state;

FIG. 14 is an exemplary signal obtained from measuring a subject's bicep in a flexion state;

FIGS. 15A to 15E show exemplary signals corresponding to various MAS values;

FIG. 16 is a bar chart of test data showing correspondence to MAS values;

FIG. 17 is a scatter plot of test data;

FIG. 18 is a schematic view of a subject's bicep moving from a flexion state to a transition state, and from the transition state to an extended state; and

FIG. 19 shows an exemplary signal acquired when the subject performs the movement illustrated in FIG. 18.

### DETAILED DESCRIPTION

Reference throughout this specification to “one embodiment”, “another embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment. Furthermore, the described features, structures, or characteristics may be combined in any suitable manner in one or more embodiments. In the following description, numerous specific details are provided to give a thorough understanding of embodiments. One skilled in the relevant art will recognize that the various embodiments may be practiced without one or more of the specific details, or with other methods, components, materials, etc. In other instances, some or all known structures, materials, or operations may not be shown or described in detail to avoid obfuscation.

The word “exemplary” is used herein to mean “serving as an example, instance, or illustration.” Any embodiment described herein as “exemplary” is not necessarily to be construed as preferred or advantageous over other embodiments. As used herein, the singular ‘a’ and ‘an’ may be construed as including the plural “one or more” unless apparent from the context to be otherwise.

Terms such as “first” and “second” are used in the description and claims only for the sake of brevity and clarity, and do not necessarily imply a priority or order, unless required by the context. The terms “about” and “approximately” as applied to a stated numeric value encompasses the exact value and a reasonable variance, as will be understood by one of ordinary skill in the art, and the terms “generally” and “substantially” are to be understood in a similar manner, unless otherwise specified.

The terms “conform”, “in conformance”, “conforming to” between two surfaces, generally describes at least a part of a first surface in compliance/full contact with a part of a second surface. It may not necessarily mean that all of a first surface complies or is in contact with all of a second surface.

The system and method as disclosed herein advantageously provides a quick (near instantaneous) and quantitative assessment of muscle stiffness or spasticity of a subject. As used in the present disclosure, the terms “muscle spasticity” or “muscle spasticity state” can be understood to be interchangeable or to also refer to “muscle stiffness” and “a degree or state of muscle stiffness” respectively, in relation to a muscle or a muscle group. Similarly, the term “muscle stiffness or spasticity” is used herein for the sake of brevity to refer to muscle stiffness and/or muscle spasticity. A near instantaneous assessment provides relief to a patient who may experience pain during typical muscle spasticity assessment/measurement process. Further, a quantitative assessment provides higher robustness in comparison to the typical qualitative assessments from clinicians. The system and method may also be performed without the presence of medical practitioners, which provides the benefit of continuous monitoring of a subject's muscle spasticity.

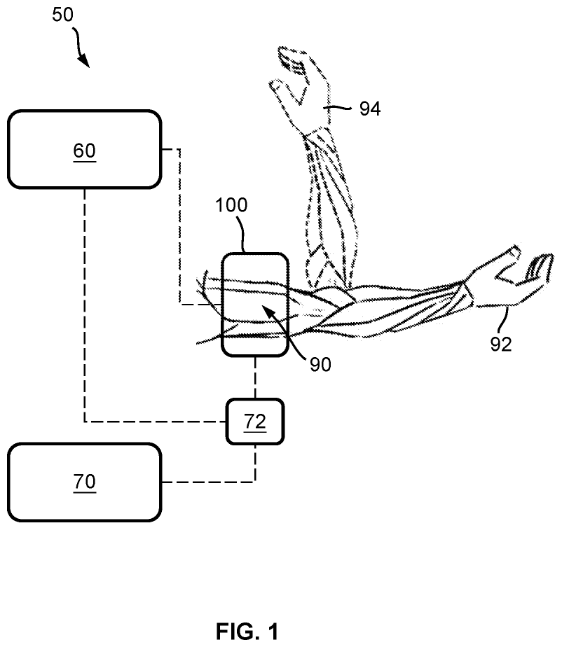

FIG. 1 illustrates an embodiment of a system 50 for testing or diagnosing a condition of a muscle or a muscle group 90 (collectively referenced as “90” for the sake of brevity). The system 50 can be operated to determine a quantitative measure corresponding to a degree of muscle spasticity of the muscle or the muscle group 90. The system 50 may include a device 100 and a controller 60 in signal communication with the device 100. In some applications, the device 100 may be positioned adjacent to the muscle or muscle group 90 of a subject, for example, on a bicep of the subject. The system 50 may be used in various tests, e.g., with the subject's arm stretched out and/or bent such that the muscle or the muscle group 90 is in an extended state 92 or a flexion state 94, respectively. The system 50 may further include a fluid source 70 in fluid communication with the device 100 through a fluid flow control system 72, such as one or more valves. The term “fluid” refers to matter in either the gaseous state or the liquid state, and in various embodiments may be used interchangeably with one or more of the following terms: “air”, “gas”, “liquid”, “pneumatic”, “hydraulic”, etc. The controller 60 may further be configured to be in signal communication with the fluid flow control system 72 to open or close the fluid communication path between the fluid source 70 and the device 100. In preferred embodiments, the term “fluid flow” can be “air flow” and the “fluid source” can be a “pneumatic source”.

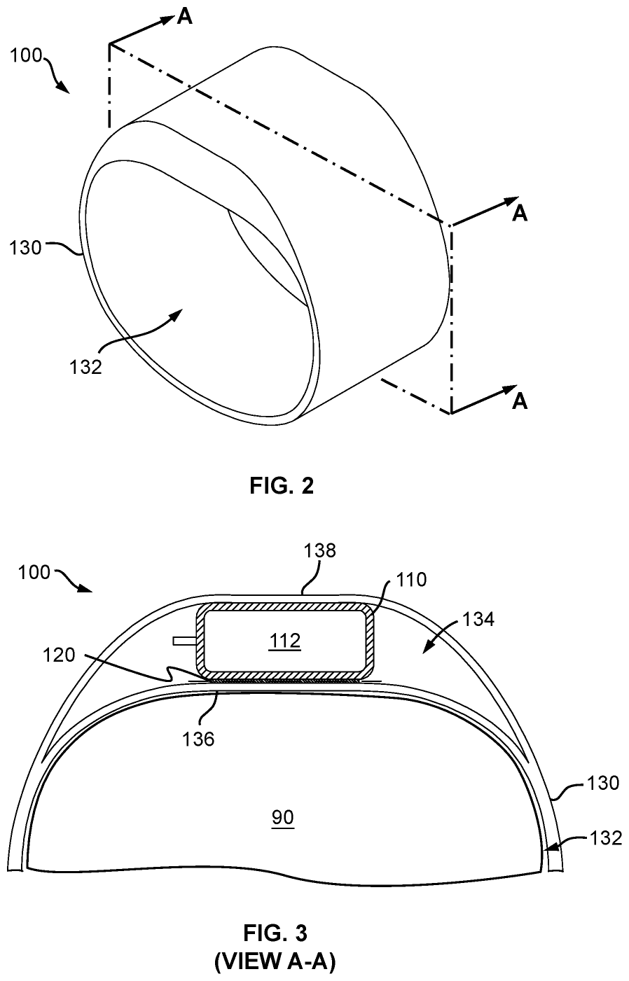

FIGS. 2 to 5 illustrate different views of the device 100 according to one embodiment of the present disclosure. As shown in FIG. 2, the device 100 may include a wearable article 130 (for example, a cuff, a bandage, or a wearable arm strap, etc.) that provides an opening 132 for receiving an arm of a subject. The wearable article 130 may be made of soft and deformable materials to ensure intimate contact with the body part/muscle/muscle group 90. The wearable article 130 may be adaptable to different body parts or in different sizes. The wearable article 130 may be made of a stretchable textile so that it can position the device in a generally fixed position relative to the relevant body part. Preferably, the wearable article 130 firmly binds or bias the device 100 against a target muscle or muscle group 90.

The device 100 includes an actuator 110 and a piezoelectric film 120. The cross-sectional view of FIG. 3 shows the wearable article 130 wrapped around a body part in proximity to a muscle or muscle group 90. The wearable article 130 includes a pocket 134 in which the actuator 110 and the piezoelectric film 120 can be disposed and held in a generally fixed position relative to the body part, inside the pocket 134. The actuator 110 and the piezoelectric film 120 may be stacked in the pocket 134 such that the piezoelectric film 120 is sandwiched between the actuator 110 and a proximal wall of the pocket 134. Preferably, the wearable article 130 does not directly press on the piezoelectric film 120. In the example shown in FIG. 3, a distal wall of the pocket 134 does not directly contact or apply a force on the piezoelectric film 120. The piezoelectric film 120 is held in place solely by an abutment of a lateral surface 116 of the actuator 110 against the piezoelectric film 120. The actuator 110 even in a relaxed state provides the lateral surface 116 in abutment with the piezoelectric film 120, i.e., the piezoelectric film 120 is biased towards the muscle or the muscle group. Preferably, the wearable article 130 is made of an elastic material and stretchable, such that when worn by the subject, the wearable article 130 pushes the actuator 110 towards the piezoelectric film 120 regardless of the state of the actuator 110, e.g., regardless of whether the actuator 110 is in a pressurized state or a relaxed state. That is, in either or both the pressurized state or the relaxed state, the actuator 110 provides the lateral surface in biased abutment with the piezoelectric film 120.

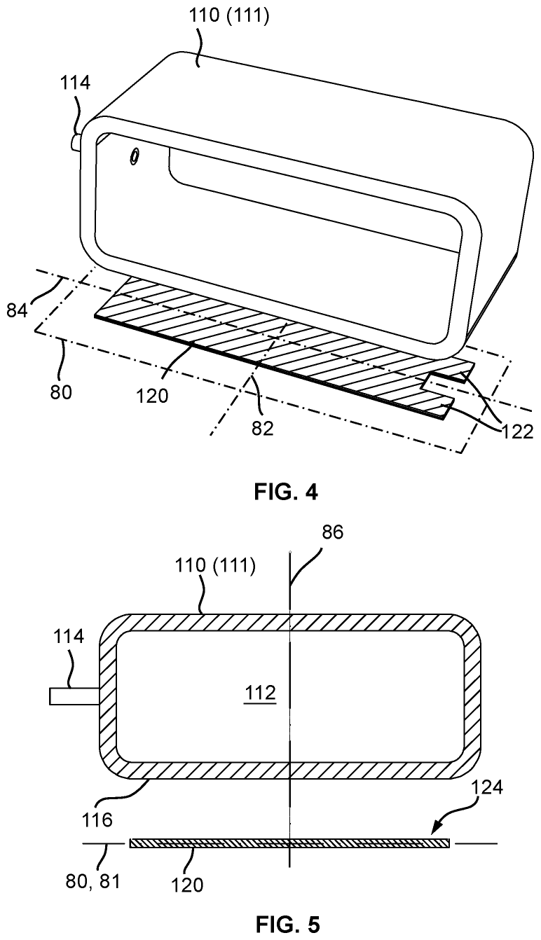

The exploded views of FIGS. 4 and 5 more clearly illustrate the relative positions of the actuator 110 and the piezoelectric film 120. The actuator 110 may include an elastic bag 111 defining a fluid cavity 112 with a variable bag volume. The lateral surface 116 may be part of the elastic bag 111. In some embodiments, the lateral surface 116 may be disposed adjacent to and in contact with the piezoelectric film 120. In some examples, the lateral surface 116 may be attached to the piezoelectric film 120. The lateral surface 116 may be larger than a film surface 124 of the piezoelectric film 120. In one example, a lateral dimension of the lateral surface 116 along a first axis 82 or along a second axis 84 may be larger than the corresponding dimensions of the piezoelectric film 120. In a non-limiting example, the lateral surface 116 has lateral dimensions larger than the corresponding dimensions of the piezoelectric film 120 by a margin of about 1 mm (millimeter) to about 2 mm all around. When the device 100 is in use, the wearable article 130 may exert a holding force on the body part and hence on the muscle or the muscle group 90 in order to attach device to the subject. The piezoelectric film 120 is configured to operate independently of the holding force, e.g., there is no direct application of the holding force on the piezoelectric film 120. The actuator 110, or more specifically the deformable elastic bag 111, serves to prevent a direct application of the holding force on the piezoelectric film 120.

The elastic bag 111 may be made of an elastomer to form a fluid cavity 112 defining a variable bag volume. The elastomer may be any one or more selected from the group including, but not limited to, polysiloxanes, polyurethanes, latex, acrylic elastomers, and styrenebutadiene elastomers. In one example, the precursors of Ecoflex 00-30 (a commercially available polysiloxane) are uniformly mixed and poured into respective negative molds. After curing, the resulting elastomeric parts are removed from the molds. The elastomeric parts may be assembled using adhesives to form the fluid cavity 112. The elastic bag 111 may be provided with a cannula or a port (hereinafter, generally referred to as a port 114), via which a fluid (such as air or another suitable fluid) may be supplied to or drawn out from the fluid cavity 112. In some embodiments, a fabric with a higher elastic modulus may be impregnated in the elastomeric parts to prevent overexpansion of the fluid cavity 112.

The fluid flow control system 72 couples the fluid source 70 with the fluid cavity 112 via the port 114. The fluid flow control system 72 is switchable between pumping a fluid into the elastic bag 111 (providing the elastic bag 111 in a pressurized state) or drawing the fluid out of the elastic bag 111 (providing the elastic bag in a relaxed state), and the elastic bag 111 is correspondingly switchable between a pressurized state and a relaxed state. For the purpose of the present disclosure, reference to the actuator 110 being in the pressurized state or in the relaxed state is interchangeable with reference to the elastic bag 111 being in the pressurized state or in the relaxed state, respectively. Depending on the choice of the material used for making the elastic bag 111, it will be understood that even in a fully relaxed state or a partially relaxed state, the elastic bag 111 may not appear fully deflated. The shape and size of the elastic bag 111 in the pressurized state and in the relaxed state may not appear to be significantly different. In some embodiments, the pressurized state may be defined by the elastic bag 111 undergoing inflation, and the relaxed state is defined the elastic bag 111 undergoing deflation. The actuator 110 may be described as being in a partially relaxed state during or after a stage of drawing the fluid out of the elastic bag 111. For the avoidance of doubt, it is not required for a vacuum to be formed in the elastic bag 111 when the actuator 110 is in the relaxed state.

Still referring to FIGS. 4 and 5, the piezoelectric film 120 may be configured as a flexible thin film, such as a polymeric-PVDF-based film. The piezoelectric film 120 may define a neutral plane 80. The neutral plane 80 is a conceptual plane in the piezoelectric film 120 where the material of the piezoelectric film is not experiencing bending stresses (tensile stress and/or compressive stress). The neutral plane 80 is present regardless of the bending state or curvature. Therefore, in the process of bending the piezoelectric film 120, the neutral plane 80 deforms according to the varying bending stresses and/or the curvature of the piezoelectric film 120. In various embodiments, the piezoelectric film 120 may have a higher tension stiffness along the neutral plane 80 and a lower bending stiffness about a bending axis 82/84 in the neutral plane 80. In other words, the piezoelectric film 120 may be more bendable or more flexible relative to its ability to stretch (in the neutral plane). In FIG. 5, the neutral plane 80 is shown as a substantially flat plane but one of ordinary skill in the art will appreciate that the neutral plane 80 is not necessarily a flat plane and that the neutral plane 80 will change with bending of the piezoelectric film 120.

Additionally, a reference plane 81 may be defined by the shape or configuration of the piezoelectric film 120 when the device 100 is positioned for operation but when the device is not in an operating mode. That is, if the device is in a non-operating mode, the reference plane 81 substantially coincides with the neutral plane 80, whereas if the device is in an operating mode, there will be instances when the neutral plane 80 of the piezoelectric film 120 is bent or displaced or otherwise deformed relative to the reference plane 81. For the purpose of the present disclosure, reference to a deformation or to at least one deformation of the piezoelectric film 120 is to be understood as a deformation relative to the reference plane 81. In other words, deformation of the piezoelectric film 120 is considered relative to the shape or configuration of the piezoelectric film 120 when the device 100 is positioned for testing but when the device is not in an operating mode.

The piezoelectric film 120 may be sandwiched by a pair of electrodes 122 or electrical contacts to provide signal communication with the controller 60. The electrodes 122 may be made of compliant conductors including but not limited to, metallic thin film, metallic-nanowire networks, carbon-nanotube networks, and conductive polymers. In some embodiments, the piezoelectric film 120 may be configured in a rectangular or circular shape to render either isotropic or anisotropic measurements. In one example, the lateral dimension of the piezoelectric film 120 may range from a few millimeters to a few centimeters. When subjected to a mechanical deformation, the piezoelectric film 120 generates a voltage in response to the change in dipole moment. Charge redistribution induced by such a voltage is collected by the electrodes 122 and further provides a voltage output in a closed circuit, i.e. such that a signal (electrical voltage or current) provided by the piezoelectric film 120 is received by the controller 60.

In some embodiments, when the elastic bag 111 is in the pressurized state, the actuator 110 deforms the neutral plane 80 of the piezoelectric film 120 such that a curvature 120a/120b of the piezoelectric film 120 conforms with both the contour of the lateral surface 116 and a local contour 91b of the muscle. Such a deformation is resisted by the adjacent muscle. A healthy muscle under relaxed status is soft and allows for large deformation, whereas a diseased muscle subjected to spasticity would provide a larger resistance to the deformation of the piezoelectric film 120. As such, a high voltage output above a certain threshold may suggests a healthy condition of the muscle or the muscle group 90, while a lower voltage output may suggest a diseased condition or a higher degree of muscle spasticity. The amplitude and waveform of such a voltage output is found to be relatively consistent (for a given device 100) such that the voltage output of the device 100 can be benchmarked to aid future determination of the severity of muscle spasticity. It may be appreciated that the curvature 120a/120b of the piezoelectric film 120 or in other cases the change in curvature (from 120a to 120b) of the piezoelectric film 120, corresponds to or is determined by a stiffness of the muscle or the muscle group 90.

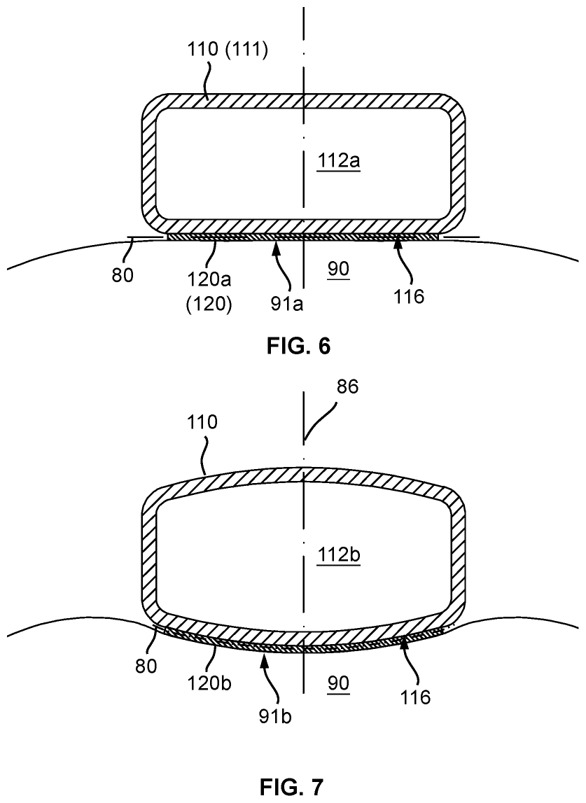

Exemplary examples of the device 100 when provided against on a subject's muscle are shown in FIGS. 6 and 7, with the wearable article 130 omitted for the sake of clarity. FIG. 6 illustrates the elastic bag 111 in the relaxed state 112a, while FIG. 7 illustrates the elastic bag 111 in the pressurized state 112b. In some embodiments, when the elastic bag 111 is in the relaxed state, or in other words at least partially deflated, the piezoelectric film 120 may conform to both a contour of the lateral surface 116 and a local contour 91a of the muscle. In the present disclosure, reference to the piezoelectric film 120 being conformable to a local contour 91a of the muscle includes reference to at least a part of a surface of the piezoelectric film 120 being in compliance with or following the shape of at least a part of the muscle or the muscle group 90.

Preferably, the device 100 is periodically or cyclically inflated and deflated to produce a signal from the piezoelectric film 120 which corresponds to a quantitative measure of a muscle spasticity state of the muscle or the muscle group 90. The actuator is controlled to periodically change between the pressurized state and the relaxed state to provide a cyclic force contributing to at least one deformation of a piezoelectric film. The cyclic force refers to a force applied by the actuator on the piezoelectric film in which the magnitude of the force varies in a cyclical manner over time. That is, the cyclic force is controllably applied to alternately and/or periodically increase in magnitude and decrease in magnitude. The actuator is configured to apply a force of greater magnitude when the actuator is in the pressurized state, and the actuator is configured to apply a force of smaller magnitude when the actuator is in the relaxed state. The cyclic force is applied concurrently with the acquisition of the signal generated by the piezoelectric film. The piezoelectric film is sandwiched between the actuator and the muscle or muscle group under testing, such that the piezoelectric film may be pressed on opposing surfaces concurrently by the actuator and by the muscle or muscle group, in which the pressing force by the actuator is the cyclic force. In both the relaxed state and the pressurized state, a lateral surface of the actuator is in abutment with the piezoelectric film to enable variable deformation and conformance of the piezoelectric film to the shape of the muscle or the muscle group. In other words, the actuator 110/elastic bag 111 cycles between the relaxed state 112b and the pressurized state 112b to produce a signal with a series of peaks. The signal is generated by deformation of the piezoelectric film, in which a change in the signal corresponds to at least one deformation of the piezoelectric film. The signal may be used for determining a condition of the muscle or the muscle group 90. For example, a quantitative measure of a muscle spasticity state of the muscle or the muscle group may be determined based on the signal. It is worth noting that the elastic bag 111 need not be fully inflated nor fully deflated in the process, beneficially reduces the duration of measurement. In other words, the elastic bag 111 is not expanded to its limit nor collapsed to its minimum size.

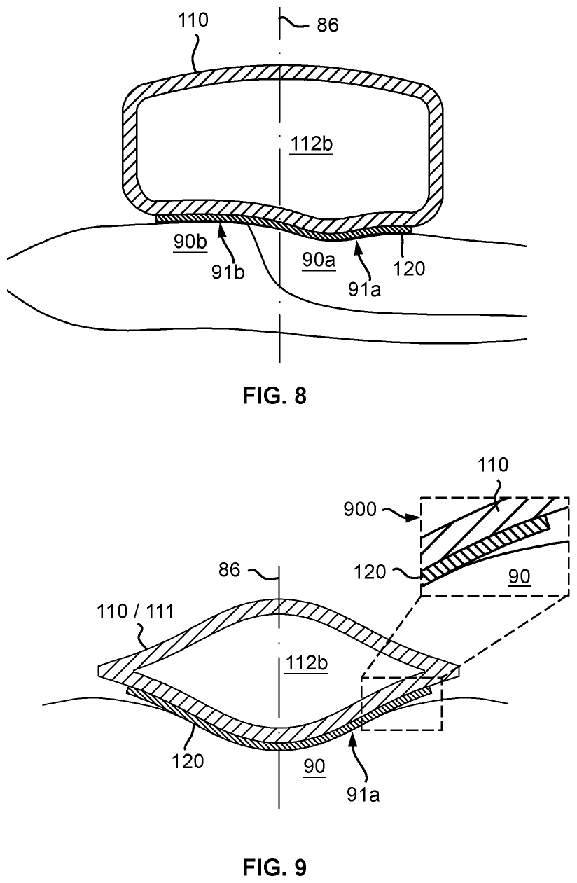

In another embodiment as illustrated in FIG. 8, the device 100 may be worn over a muscle group 90 comprising a plurality of muscles 90a/90b. It may be appreciated that due to different muscle stiffnesses or muscle spasticity, with the actuator 110 in the pressurized state 112b, the piezoelectric film 120 may deform and conform differently over different muscles 90a/90b. In an example illustrated in FIG. 8, a portion/part of the piezoelectric film 210 conforms with a first local contour 91a of a muscle 90a, and another portion/part of the piezoelectric film 210 conforms with a second local contour 91b of a muscle 90b. In this example, muscle 90b has a higher stiffness in comparison to muscle 90a. Therefore, the signal acquired from the piezoelectric film 120 comprises a deformation corresponding to both muscle 90a and muscle 90b. In other words, the piezoelectric film is conformable to both the actuator in the pressurized state and to the muscle or the muscle group.

FIG. 9 illustrates the elastic bag 111 of the actuator 110, in accordance with another embodiment of the device 100. The elastic bag 111 may include a wider center portion with narrowed ends as illustrated in FIG. 9. In this embodiment, as shown by the magnified view 900, only a portion of the piezoelectric film 120 may conform to a local contour 91a of the muscle. The piezoelectric film 120 is flexible and variably deformable, i.e., deformable in different ways across the area of the lateral surface, in response to a stiffness of the muscle or the muscle group.

The thickness of the elastic bag 111 may be less than 5 mm to allow the device 100 to conform to muscle/muscle group 90 intimately. The elastic bag 111 inflates in a thickness direction 86 when a fluid is pumped in, such that the elastic bag 111 is in the pressurized state 112b. The elastic bag 111 returns to the relaxed state 112a when the fluid is vented from the elastic bag 111. The inflation and deflation of the actuator 110/elastic bag 111 is rapid due to the low viscosity of the fluid, thereby allowing a quick periodically change between the pressurized state 112b and the relaxed state 112a. In other words, the device 100 is configured to enable a relatively high frequency of change between the pressurized state and the relaxed state 112a/112b. In some examples, the fluid pressure supplied to the actuator 110/elastic bag 111 determines the degree in which the neutral plane 80 of the piezoelectric film 120 deforms.

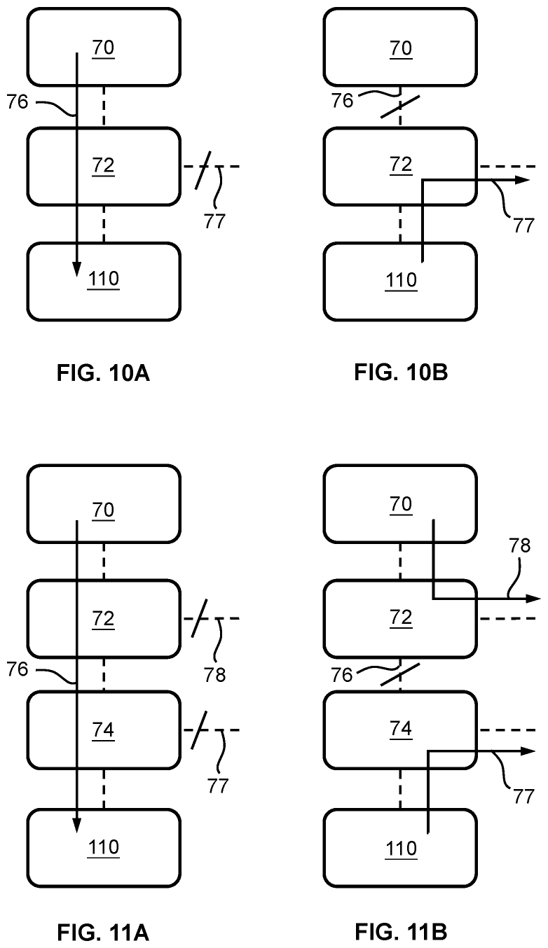

According to various embodiments, referring to FIGS. 10A and 10B, the fluid source 70 may include a pump in fluid communication with the actuator 110 through a valve 72 (also interchangeably referred to as a fluid flow control system). In a preferred embodiment, the fluid can be air at an air pressure (fluid pressure) ranging from about 5 kPa (kilopascals) to about 50 kPa, with a volumetric air flow (volumetric fluid flow) ranging from about 1 L/min (liter per minute) to about 20 L/min. The valve 72 may be a 3/2-way (three ports, two flow paths) design to allow both actuating (FIG. 10A) and venting (FIG. 10B) of the actuator 110. In some embodiments, the valve 72 may be an electrically controlled solenoid valve which can switch between two positions 60, an active position as shown in FIGS. 10A and a rest position as shown in 10B, driven by an electric current supplied by the controller 60. The active position opens path 76 and shuts path 77 such that the actuator 110 is inflated or pressurized by the pump 70 to assume the pressurized state. The rest position functions reversely with path 78 opened and path 76 is closed, such that the actuator 110 may vent and assume the related state. If fluid flow (from the pump 70) and fluid supplying time (determined by the controller 60 via the valve) are both pre-determined, the specific amount of fluid to be fed into the actuator 110 can be determined. In another embodiment as shown in FIGS. 11A and 11B, an additional 3/2-way valve 74 may be provided in series with the valve 72. This allows the fluid to escape from the actuator 110 from the valve 74 when the valve 72 is in the rest position. This prevents over pressurization in the path between the pump/fluid source 70 and the valve 72 (FIG. 11B).

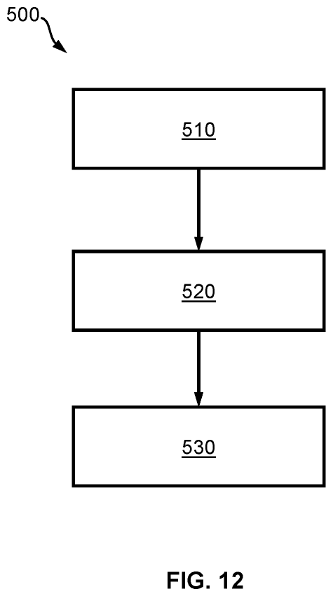

According to another aspect, a diagnostic method for a condition of a muscle or a muscle group 90 is disclosed. The method may be implemented or carried out by use of the system 50 and device 100 as described above. Referring to FIG. 12, a method 500 of diagnosing a condition of a muscle or a muscle group 90, such as a muscle spasticity, is disclosed. The method 500 comprises controlling an actuator to periodically change between a pressurized state and a relaxed state (510). The actuator in the relaxed state provides a lateral surface in biased abutment with a flexible piezoelectric film. The piezoelectric film may be disposed between the lateral surface and the muscle or the muscle group 90. When the actuator is in the pressurized state deforms a neutral plane of the piezoelectric film such that a curvature of the piezoelectric film conforms with a contour of the lateral surface of the actuator and a local contour of the muscle or the muscle group 90. It may be appreciated that the curvature of the piezoelectric film or in other cases the change in curvature of the piezoelectric film, corresponds to or is determined by the muscle/muscle group stiffness.

In some embodiments, the method 500 includes controlling a fluid communication between a fluid source and an elastic bag of the actuator. The method 500 further includes acquiring a signal generated by the piezoelectric film (520). The signal corresponds to a change in the curvature of the piezoelectric film. The signal is being generated concurrently with the actuator changing between the pressurized state and the relaxed state. In some embodiments, the signal may be used to determine at least one of the following: (i) at least one local peak (flexion) 320 of the signal, (ii) a mean of plurality of local peak (flexion) 320 of the signal, (ii) a ratio between at least one local peak (flexion) 320 of the signal and at least one local peak (extended) 310 of the signal, (iii) or any combination thereof.

In some embodiments, the actuator 110 is partially inflated and partially deflated in the course of periodically changing between the pressurized state and the relaxed state. This beneficially provides a quick determination of the subject's muscle spasticity state as there is no need to fully inflate and deflate the actuator, but to only provide an alternating compression/relaxation cycle.

In some embodiments, periodically changing between the pressurized state and the relaxed state is controlled by the controller 60 with precision control over on (inflation) time and off (deflation) time. The periodic/cyclic inflation and deflation period may be tuned by controlling the valve 72/74 to meet a threshold frequency range. Therefore, actuator 110 is configured to switch between the pressurized state and the relaxed state at a frequency within a frequency range. In an embodiment, the frequency range may be less than 8 Hz, preferably between 2 Hz to 8 Hz. By switching between the pressurized state and the relaxed state within the frequency range, a sufficient voltage output may be produced by the piezoelectric film. According to an example, when the input pressure was fixed at 30 kPa, the actuator was cycled at different period/frequency between the pressurized state and the relaxed state. It was found that 0.1 second period is insufficient to reach optimal voltage output, while 0.2 second period readily produces sufficient voltage output (about 8V output). Further increasing the period (decreasing the frequency) did not result in an increase in the voltage output. Therefore, a period between 0.2 seconds to 0.6 seconds may be a suitable range.

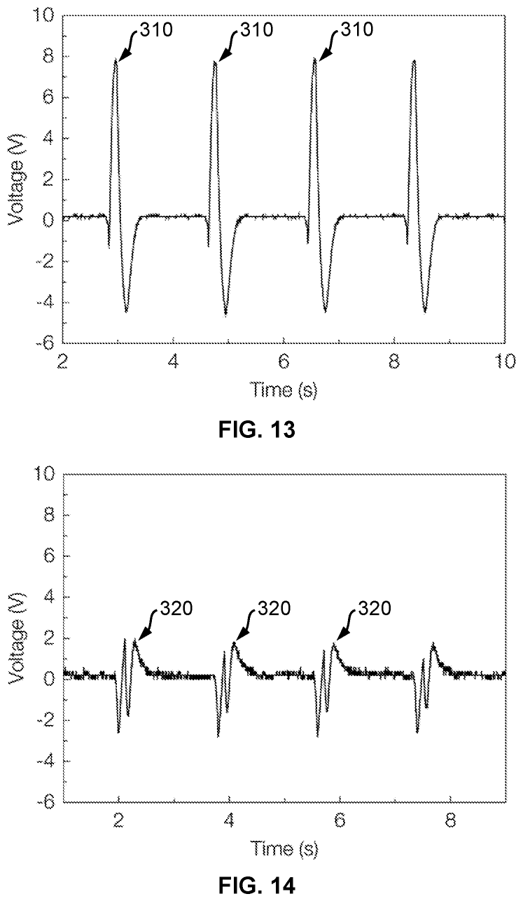

According to some embodiments, the method may be carried out in a “static mode” or when the muscle/muscle group 90 is in a static state, whereby a subject remains still with his/her arm in an extended state 92 (FIG. 1). The relaxed biceps of a healthy subject are soft and deformable, thus imposing little restriction to the deformation/bending of the piezoelectric film. As such, the piezoelectric film may bend to a large extend upon the actuator in a pressurized state, and registering a prominent peak of positive voltage as shown in FIG. 13. In subjects with muscle spasticity, the biceps may be stiff and less deformable, thus imposing a relatively higher restriction to the deformation/bending of the piezoelectric film. When the actuator vents and ceases the bending/compressive motion on the piezoelectric film, the piezoelectric film will relax from bending and register a negative voltage peak following the positive one.

In some embodiments, the signal received from the piezoelectric film may be a time series voltage signal comprising at least one local peak corresponding to the actuator in the pressurized state. In some examples, the inflation and deflation of the actuator may be performed periodically when the bicep is in the extended state 92, thus generating a series of local peaks (extended) 310. In some embodiments, a mean amplitude of the series of local peaks (extended) 310 corresponds to a degree of severity of the muscle spasticity state.

Further, the “static mode” may be performed when the subject tense his/her biceps, such as assuming the flexion state 94 (FIG. 1), to render higher muscle stiffness. In some embodiments, the method may be carried out when the muscle/muscle group 90 is in an isometric contraction. As a stiffer muscle provides higher restriction to deformation of the piezoelectric, the amplitude of voltage output is reduced, as shown in FIG. 14. Similarly, the inflation and deflation of the actuator may be performed periodically when the bicep is in the flexion state 94, thus generating a series of local peaks (flexion) 320.

In some embodiments, the “static mode” may be performed when the subject's arms are held static in the extended state 92 and held static in the flexion state 94. Therefore, the signal collected may include the series of local peaks (flexion) 320 corresponding to when the muscle or the muscle group 90 is in the flexion state and the series of local peaks (extended) 310 corresponding to when the muscle or the muscle group 90 in the extended state. Further, the muscle spasticity state may be determined based on a ratio between at least one local peak (flexion) 320 and the at least one local peak (extended) 310. In another embodiment, the muscle spasticity state may be determined based on a difference between at least one local peak (flexion) 320 and the at least one local peak (extended) 310.

The method 500 may further include determining a quantitative measure of a muscle spasticity state of the muscle or the muscle group based on the signal. The method may involve determining the quantitative measure based on at least one of the following: (i) at least one local peak of the signal, (ii) a mean of the at least one local peak of the signal, and (iii) a ratio between at least one first local peak of the signal and at least one second local peak of the signal. The method 500 may distinguish between different levels of muscle stiffness and correlate the respective levels to the severity of spasticity, such as providing a quantitative measure that correlates to a Modified Ashworth Scale (MAS) value. In some examples, the method 500 may further involve determining a correlation table based on at least one of the following: (i) at least one local peak of the signal, (ii) a mean of plurality of local peak of the signal, (ii) a ratio between at least one first local peak of the signal and at least one second local peak of the signal, (iii) or any combination thereof.

The practical use and benefits of the present method and device were verified in clinical applications. In one example, the device 100 was attached around an upper arm of a subject with the actuator and piezoelectric film provided on the biceps. A circular gel electrode may be used to ground the human body to reduce signal noise. The subject, lying on a bed, was asked not to voluntarily control the muscles. Spasticity in the upper limb usually causes increased muscle tone (typically in biceps) and locks the arm in a bent/flexed position or flexion state. Biceps stiffness was evaluated under both flexion state and extended state. Such measurements may be considered as “static mode” testing, i.e., the muscle/muscle group 90 is kept still during the evaluating process.

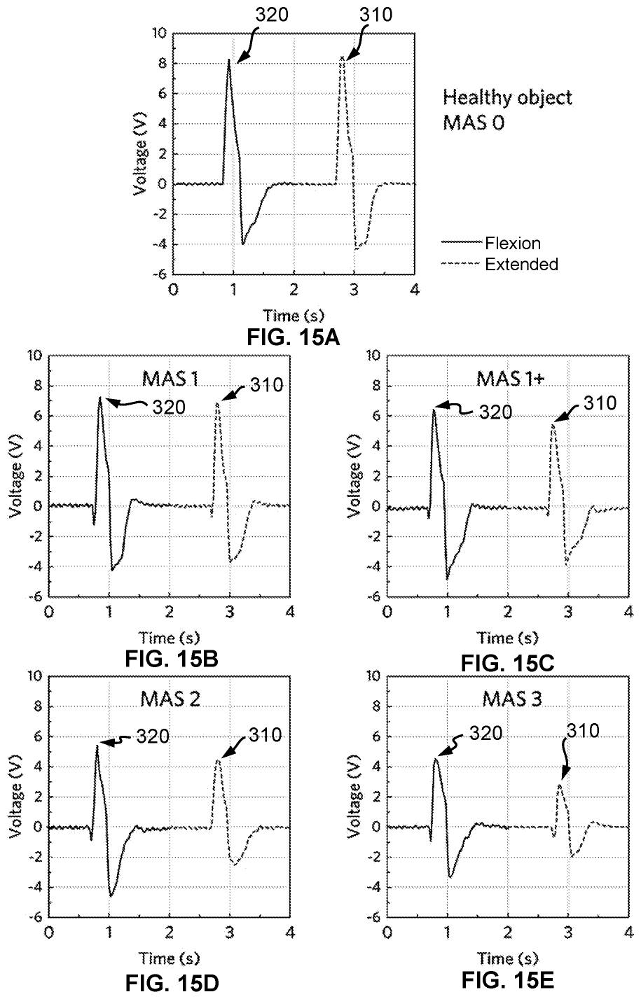

Referring to FIG. 15, muscle stiffness measurements refer to the amplitude of the voltage output from the piezoelectric film. With arms in a flexion state, the integrated diagnostic system read about 8 V (volts) voltage output from healthy volunteers, and gradually reduced voltage output from spasticity patients (e.g., about 4 V from MAS 3 patients). A clear decline in the voltage reading was observed as the severity of muscle spasticity increased (FIGS. 15A to 15E, solid lines 320). Measurements of muscle stiffness were further performed with the arms bent and held in the extended state. For healthy volunteers, the wearable device gave similar reading (about 8V) while measuring under flexion and extension conditions because the biceps was always relaxed no matter which posture it is being posed. On the contrary, a spastic muscle would resist the elbow bending process imposed by the clinician, manifesting increased muscle tone and a lower voltage readout when the arm was held in extension position (FIGS. 15A to 15E, dashed lines 310).

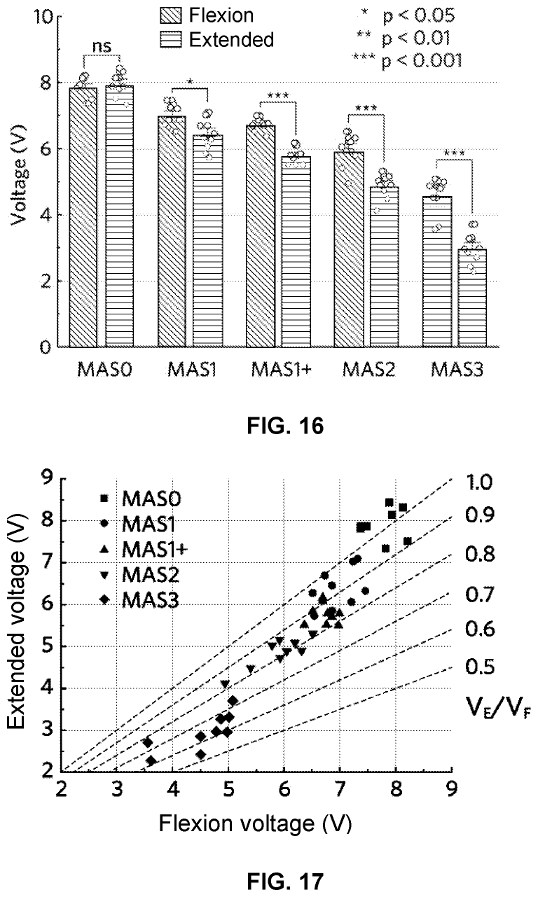

The measurements collected from the above example are summarized in FIG. 16, with the results correlated with the MAS values diagnosed by a clinician. The flexion voltages and extended voltages collected from various healthy subjects are statistically non-significant, while those collected from patients show statistical significance (which means the flexion voltages and extended voltages are statistically different in their average mean and distribution). The severity of spasticity could further be predicted by comparing the difference, or ratio, between extended voltages and flexion voltages (VE/VF). By referring to the trendlines in FIG. 17, it can be observed that the data points of healthy volunteers distribute around VE/VF=1.0, while the data points collected from subjects with muscle spasticity (MAS 1, 1+, 2 and 3) move towards lower VE/VF value as spasticity becomes more severe.

In the static mode, the method 500 and system 50 offer the capability to evaluate spasticity from two aspects: (i) to directly read the absolute voltage output measured from flexion positions to interpret the nominal stiffness of spastic muscles (when they are postured at their naturally locked, flexed position); and/or (ii) read the ratio between extended voltages and flexion voltages to understand the change in muscle tone when the spastic muscle is subjected to bending.

According to another embodiment, the method 500 of testing in a dynamic testing mode for a condition of a muscle or a muscle group 90 is disclosed. The method 500 may be referred to as a “dynamic mode” test if the subject's arm is passively extended by a clinician from a maximal possible flexion (flexion state 94) to a maximal possible extension (extended state 92). In the dynamic testing mode, the actuator 110 is controlled to activate (deform) the piezoelectric sensor 120 periodically at a background frequency (5 Hz) to generate a background waveform. The amplitude of the voltage wave is highly sensitive to the subtle change in muscle stiffness, therefore the device 100 can effectively assess how much the muscle tone increases, and how long the bending process lasts. The method 500 may involve (i) periodically switching the actuator 100 between the pressurized state and the relaxed state; and (ii) concurrently acquiring the signal from the piezoelectric film 120 when the muscle is in a transition state. Referring to FIG. 19, a transition state 96 is being characterized by a dynamic motion of the muscle/muscle group 90 between a flexion state 94 of the muscle/muscle group 90 and an extended state 92 of the muscle/muscle group 90.

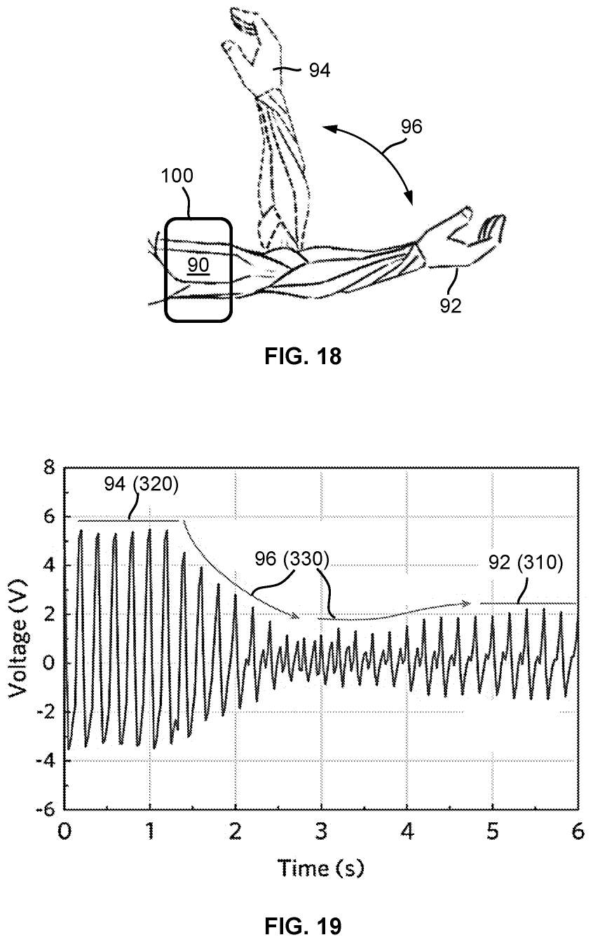

In an example as shown in FIG. 18, when performing dynamic mode measurement on the biceps of a MAS 3 patient, a plateau (at flexion state) was firstly recorded before arm bending. The onsite of bending is accompanied with a significant drop in voltage reading (from about 5.5 V to about 1 V), suggesting a substantial increment in muscle stiffness as the diseased muscle was resisting the external force and rendering increased muscle tone during the period of about 2 seconds of bending motion. Thereafter, the patient's arm was held at the extended state and the signal shows a gradual recovery in baseline voltage, which stabilizes at about 2V (defined as plateau of extension). Such result reveals that a spastic muscle is likely to respond to a rapid elbow bending with an overly increased muscle tone, they gradually adapt to the stress and render a slightly lower muscle tone.

Referring to FIG. 19, the signal obtained from the piezoelectric film for determining the quantitative measure of the muscle spasticity state is a continuous time series signal (a signal including a series of peaks over time) which includes at least one local peak (flexion) 320 corresponding to the flexion state 94, at least one local peak (extended) 310 corresponding to the extended state 92, and at least one local peak (transition) 330 corresponding to the transition state 96. Departing from intuition, the at least one local peak (transition) 330 is characterized by a smaller voltage value than each of the at least one local peak (extended) 310 and the local peak (flexion) 320. In an embodiment, a difference between the at least one local peak (transition) 330 and the at least one local peak (flexion) 320 corresponds to the muscle spasticity state.

All examples described herein, whether of apparatus, methods, materials, or products, are presented for the purpose of illustration and to aid understanding and are not intended to be limiting or exhaustive. Various changes and modifications may be made by one of ordinary skill in the art without departing from the scope of the invention as claimed.

1. A method of testing a muscle or a muscle group for muscle stiffness or spasticity, the method comprising:
controlling an actuator to periodically change between a pressurized state and a relaxed state such that the actuator in at least the pressurized state provides a cyclic force contributing to at least one deformation of a piezoelectric film, the actuator in at least the relaxed state providing a lateral surface in abutment with the piezoelectric film, the piezoelectric film being variably deformable and conformable to the muscle or the muscle group;
acquiring a signal generated by the at least one deformation of the piezoelectric film, wherein a change in the signal corresponds to the at least one deformation of the piezoelectric film; and
determining a quantitative measure of a muscle spasticity state of the muscle or the muscle group based on the signal.

2. The method as recited in claim 1, wherein the piezoelectric film is conformable to both the actuator in the pressurized state and to the muscle or the muscle group.

3. The method as recited in claim 1, wherein the piezoelectric film is flexible, and wherein the piezoelectric film is variably deformable in response to a stiffness of the muscle or the muscle group.

4. The method as recited in claim 1, wherein the piezoelectric film is held in a test position independently of the cyclic force, the test position being between the lateral surface and the muscle or the muscle group.

5. The method as recited in claim 1, the method comprising:
controlling a fluid communication between a fluid source and an elastic bag of the actuator, wherein the lateral surface is part of the elastic bag, and wherein the actuator is in the pressurized state if the elastic bag is inflated by the fluid source, wherein the actuator is in the relaxed state if the elastic bag is at least partially deflated.

6. The method as recited in claim 1, wherein the signal is a time series voltage signal comprising at least one local peak corresponding to the pressurized state.

7. The method as recited in claim 6, wherein a mean amplitude of the at least one local peak corresponds to a degree of severity of a muscle spasticity state.

8. The method as recited in claim 1, wherein the signal is acquired when the muscle or the muscle group is in one of an isometric contraction and a static state.

9. (canceled)

10. The method as recited in claim 1, wherein the signal includes at least one first local peak if the muscle or the muscle group is in a flexion state, and wherein the signal includes at least one second local peak if the muscle or the muscle group is in an extended state.

11. The method as recited in claim 10, the method comprising: determining the muscle spasticity state based on a ratio between the at least one first local peak and the at least one second local peak.

12. The method as recited in claim 10, the method comprising: determining the muscle spasticity state based on a difference between the at least one first local peak and the at least one second local peak.

13. The method as recited in claim 1, the method further comprising: determining a correlation table based on at least one of the following: (i) at least one local peak of the signal, (ii) a mean of plurality of local peak of the signal, (ii) a ratio between at least one first local peak of the signal and at least one second local peak of the signal, (iii) or any combination thereof.

14. (canceled)

15. The method as recited in claim 1, further comprising: periodically switching the actuator between the pressurized state and the relaxed state; and concurrently acquiring the signal from the piezoelectric film when the muscle is in a transition state, the transition state being characterized by a dynamic motion of the muscle or the muscle group between a flexion state of the muscle and an extended state of the muscle.

16. (canceled)

17. The method as recited in claim 15, wherein the signal comprises a time series of peaks, the signal including at least one first local peak corresponding to the flexion state, at least one second local peak corresponding to the extended state, and at least one third local peak corresponding to the transition state.

18. The method as recited in claim 17, wherein the at least one third local peak is characterized by a smaller voltage value than each of the at least one first local peak and the at least one second local peak.

19. The method as recited in claim 17, wherein a difference between the at least one third local peak and the at least one first local peak corresponds to a muscle spasticity state.

20. A device for testing a muscle or a muscle group for muscle stiffness or spasticity in accordance with the method as recited in claim 1, the device comprising:
a piezoelectric film; and
an actuator, the actuator in at least a relaxed state having a lateral surface in abutment with the piezoelectric film, the actuator being operable to periodically change between a pressurized state and the relaxed state such that the actuator in at least the pressurized state provides a cyclic force contributing to at least one deformation of the piezoelectric film, the piezoelectric film being variably deformable and conformable to the muscle or the muscle group, wherein the at least one deformation of the piezoelectric film is configured to generate a signal, and wherein a change in the signal corresponds to the at least one deformation of the piezoelectric film.

21. (canceled)

22. The device as recited in claim 20, wherein the piezoelectric film is flexible, and wherein the piezoelectric film is variably deformable in response to a stiffness of the muscle or the muscle group, and wherein the piezoelectric film is configured to provide a first signal representative of a first curvature change of the piezoelectric film when the muscle is in flexion and a second signal representative of a second curvature change of the piezoelectric film when the muscle is in extension.

23. (canceled)

24. The device as recited in claim 20, further comprising a wearable article, wherein the piezoelectric film is held by the wearable article in a test position independently of the cyclic force, the test position being between the lateral surface and the muscle or the muscle group, and wherein the piezoelectric film has a higher tension stiffness along a neutral plane of the piezoelectric film, and wherein the piezoelectric film has a lower bending stiffness about a bending axis in the neutral plane of the piezoelectric film.

25. (canceled)

26. (canceled)

27. (canceled)

28. A system for performing the method of testing a muscle or a muscle group for muscle stiffness or spasticity as recited in claim 1, the system comprising:
a wearable article;
a piezoelectric film;
an actuator, the actuator including a lateral surface, the actuator being attachable to the wearable article with the piezoelectric film disposed between the lateral surface of the actuator and the muscle or the muscle group, the actuator being configured to enable the piezoelectric film to be variably deformable and conformable to the muscle or the muscle group;
a controller; and
a fluid source, the fluid source being coupled to the controller, the controller being configured to open or close a fluid communication between the fluid source and the actuator to periodically change the actuator between a pressurized state and a relaxed state,
wherein the controller is configured to:
control the actuator to periodically change between the pressurized state and the relaxed state, the actuator in at least the pressurized state providing a cyclic force contributing to at least one deformation of the piezoelectric film, the actuator in at least the relaxed state having the lateral surface in abutment with the piezoelectric film;
acquire a signal generated by the at least one deformation of the piezoelectric film, a change in the signal corresponding to the at least one deformation of the piezoelectric film; and
determine a quantitative measure of a muscle spasticity state of the muscle or the muscle group based on the signal.

29. (canceled)
